(19) United States
(12) Patent Application Publication
(10) Pub. No.: US 2018/0317811 A1
(43) Pub. Date: Nov. 8, 2018

(54) APPARATUS AND METHOD FOR DETERMINING STROKE DURING SLEEP

(71) Applicants: Yang Soo LEE, Suseong-gu, Daegu (KR); Yong Won KIM, Suseong-gu, Daegu (KR)

(72) Inventors: Yang Soo LEE, Daegu (KR); Yong Won KIM, Daegu (KR); Tae Joon PARK, Seoul (KR); Sang Hoon JEON, Daegu (KR)

(21) Appl. No.: 15/771,124

(22) PCT Filed: Oct. 28, 2016
(86) PCT No.: PCT/KR2016/012205

§ 371 Date: Apr. 26, 2018

(30) Foreign Application Priority Data
Oct. 28, 2015 (KR) 10-2015-0150111

(51) Int. Cl. A61B 5/11

(52) U.S. Cl. CPC A61B 5/1114; G16H 50/30; A61B 5/1116

(57) ABSTRACT

The present invention relates to an apparatus and a method for determining a stroke during sleep capable of determining whether or not a stroke occurs during sleep of a user (a patient). Accuracy of determination for whether or not the stroke occurs is improved by applying a sleeping position (SP) filter (a processing process of deleting a measurement result when a user is not lying with his/her breast facing upward) and then comparing a ratio between left and right motions to determine whether or not the stroke occurs.

### TECHNICAL FIELD

The present invention relates to an apparatus and a method for determining a stroke during sleep capable of determining whether or not a stroke occurs during sleep of a user (a patient).

### BACKGROUND ART

A stroke is a term used to refer to both of cerebral infarction (an ischemic stroke) caused by blockage of cerebral blood vessels and cerebral hemorrhage (a hemorrhagic stroke) caused by leak of blood into a brain tissue due to the rupture of the cerebral blood vessels, and the cerebral infarction is classified into a transient ischemic attack, cerebral infarction in large vessel disease, cerebral infarction in cardiogenic embolism, small vessel disease or lacunar infarction, and the like.

It was reported to the academic world that the probability that a stroke will occur within 48 hours after the transient ischemic attack (TIA) is 3% to 5%, and twenty-three percent of patients with lacunar infarction are deteriorated in terms of neurological symptoms within 48 hours.

Thrombolysis using a tissue plasminogen activator (TPA) is a standard treatment method of a stroke, and the earlier the time in which the thrombolysis using a TPA is performed after the stroke occurs (it is preferable that the thrombolysis using a TPA is performed within three hours after the stroke occurs), the better the progress. Therefore, early detection and early treatment of the stroke are the most important principle in the treatment of the stroke.

A ratio of strokes occurring during sleep to all strokes is about 6.4% to 20%. A patient does not feel symptoms of the stroke during sleep, and it is difficult to persons around the patient to sense whether or not the stroke during sleep occurs, such that there is a difficult in early finding occurrence of the stroke.

Therefore, the thrombolysis may not be early performed due to the difficulty in early finding the stroke, and prognosis of the symptoms of the stroke is further deteriorated.

Therefore, there is a need to monitor a patient having a high likelihood of the stroke to enable early determination of the stroke occurring during the sleep.

Patent Document 1 {Korean Patent Laid-Open Publication No. 10-2013-0105135 (published on Sep. 25, 2013)} relates to an apparatus and a method for sensing a stroke. Patent Document 1 discloses a ‘technology of sensing a stroke by comparing a difference between motions of a left-side body and a right-side body using the fact that the difference between the motions of the left-side body and the right-side body becomes large at the time of occurrence of the stroke’. However, since a case in which a difference between motions of a left-side body and a right-side body during sleep is large exists in normal persons, the apparatus and the method disclosed in Patent Document 1 are highly likely to determine that the stroke exists even in the case in which the stroke does not exist.

### DISCLOSURE

### Technical Problem

An object of the present invention is to improve accuracy in an apparatus and a method for determining whether or not a stroke during sleep occurs from a difference between left and right motions of a body.

### Technical Solution

According to an exemplary embodiment of the present invention, an apparatus for determining a stroke during sleep may include: a left-side motion measurement unit wearable on a left arm or a left leg; a right-side motion measurement unit wearable on a right arm or a right leg; a posture measurement unit wearable on a body; and a determination unit receiving outputs of the left-side motion measurement unit, the right-side motion measurement unit, and the posture measurement unit and determining whether or not the stroke occurs.

The determination unit may apply a sleeping position (SP) filter (a processing process of deleting a measurement result when a user is not lying with his/her breast facing upward), and then compare a ratio between left and right motions to determine whether or not the stroke occurs.

The determination unit may determine that the stroke occurs in the case in which the user maintains a posture other than a posture at which he/she lies on his/her back, for a predetermined time or more.

According to another exemplary embodiment of the present invention, an apparatus for determining a stroke during sleep may include: a left-side motion measurement unit wearable on a left arm or a left leg; a right-side motion measurement unit wearable on a right arm or a right leg; a posture estimation unit receiving outputs of the left-side motion measurement unit and the right-side motion measurement unit and estimating a user's posture; and a determination unit receiving outputs of the left-side motion measurement unit, the right-side motion measurement unit, and the posture estimation unit and determining whether or not the stroke occurs.

According to still another exemplary embodiment of the present invention, an apparatus for determining a stroke during sleep may include: a left-side motion measurement unit wearable on a left arm or a left leg; a right-side motion measurement unit wearable on a right arm or a right leg; and a determination unit receiving outputs of the left-side motion measurement unit and the right-side motion measurement unit and determining whether or not the stroke occurs, wherein the determination unit receives the outputs of the left-side motion measurement unit and the right-side motion measurement unit and estimates the user's posture, and then determines whether or not the stroke occurs using an estimation result.

According to yet still another exemplary embodiment of the present invention, a method for determining a stroke during sleep using an apparatus for determining a stroke during sleep including a left-side motion measurement unit, a right-side motion measurement unit, a posture measurement unit, and a determination unit may include: a step of receiving outputs of the left-side motion measurement unit, the right-side motion measurement unit, the posture measurement unit by the determination unit; a step of applying an SP filter by the determination unit; and a step of determining whether or not the stroke occurs from a result obtained by applying the SP filter by the determination unit.

According to yet still another exemplary embodiment of the present invention, a method for determining a stroke during sleep using an apparatus for determining a stroke during sleep including a left-side motion measurement unit, a right-side motion measurement unit, a posture estimation unit, and a determination unit may include: a step of receiving outputs of the left-side motion measurement unit and the right-side motion measurement unit and then estimating a user's posture, by the posture estimation unit; a step of receiving outputs of the left-side motion measurement unit, the right-side motion measurement unit, and the posture estimation unit by the determination unit; and a step of applying an SP filter by the determination unit; and a step of determining whether or not the stroke occurs from a result obtained by applying the SP filter by the determination unit.

According to yet still another exemplary embodiment of the present invention, a method for determining a stroke during sleep using an apparatus for determining a stroke during sleep including a left-side motion measurement unit, a right-side motion measurement unit, and a determination unit may include: a step of receiving outputs of the left-side motion measurement unit and the right-side motion measurement unit and then estimating a user's posture, by the determination unit; a step of applying an SP filter on the basis of an estimation result by the determination unit; and a step of determining whether or not the stroke occurs from a result obtained by applying the SP filter by the determination unit.

### Advantageous Effects

In the apparatus and the method for determining a stroke during sleep according to the present invention, accuracy of determination for whether or not the stroke occurs is higher than that of the related art.

### DESCRIPTION OF DRAWINGS

FIG. 1 is a conceptual diagram of an apparatus for determining a stroke during sleep according to the present invention.

FIG. 2 is a view illustrating an example of a form in which a user wears a left-side motion measurement unit.

FIG. 3 is a view illustrating an example of a form in which the user wears a right-side motion measurement unit.

FIG. 4 is a view illustrating an example of a form in which the user wears a posture measurement unit.

FIG. 5 is a view illustrating a form in which the user wearing the left-side motion measurement unit, the right-side motion measurement unit, and the posture measurement unit is lying.

FIG. 6 is an example of measurement result graphs of a normal person.

FIGS. 7A and 7B are views illustrating a form of comparing measurement result graphs depending on whether or not a sleeping position (SP) filter exists with each other.

### DETAILED DESCRIPTION OF MAIN ELEMENTS

10: left-side motion measurement unit 20: right-side motion measurement unit

30: posture measurement unit 40: determination unit

### BEST MODE

The present invention may be variously modified and have several exemplary embodiments. Therefore, specific exemplary embodiments of the present invention will be illustrated in the accompanying drawings and be described in detail. However, it is to be understood that the present invention is not limited to a specific exemplary embodiment, but includes all modifications, equivalents, and substitutions without departing from the scope and spirit of the present invention. When it is determined that a detailed description for any known art related to the present invention may obscure the gist of the present invention, the detailed description will be omitted.

Terms used in the present specification are used only in order to describe specific exemplary embodiments rather than limiting the present invention. Singular forms are intended to include plural forms unless the context clearly indicates otherwise.

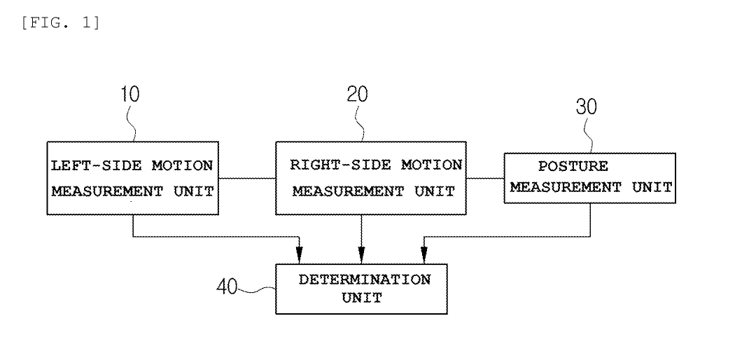

FIG. 1 is a conceptual diagram of an apparatus for determining a stroke during sleep according to the present invention.

The apparatus for determining a stroke during sleep according to the present invention includes a left-side motion measurement unit 10, a right-side motion measurement unit 20, a posture measurement unit 30, and a determination unit 40.

The left-side motion measurement unit 10 is a device measuring a motion of a left-side body (for example, a left-side leg or a left-side arm), and the right-side motion measurement unit 20 is a device measuring a motion of a right-side body (for example, a right-side leg or a right-side arm). Each of the left-side motion measurement unit 10 and the right-side motion measurement unit 20 may include an acceleration sensor (a gravity sensor) or an angular acceleration sensor (a gyro sensor) to measure a motion.

When a sensor unit is positioned at a distal end portion (for example, a wrist, an ankle, or the like) of a leg or an arm at the time of measuring the motion, it is easy to measure the motion. Therefore, it is preferable that the left-side motion measurement unit 10 is manufactured in a form in which it is wearable on the distal end portion (for example, the wrist, the ankle, or the like) of the leg or the arm.

There are several methods for manufacturing the left-side motion measurement unit in the wearable form. For example, the left-side motion measurement unit is manufactured in a form in which an electronic device is fixed to a rubber band or a long belt having a ring shape and the rubber band or the long belt may be tied to the wrist, the ankle, or the like.

Since several methods for manufacturing any device in a form in which any device is wearable on a body are known, a detailed description therefor will be omitted.

The posture measurement unit 30 is a device measuring a posture at which a user (a patient) is lying. That is, the posture measurement unit 30 determines whether the user (the patient) is lying on his/her back (is lying with his/her breast facing upward), is lying on his/her right side, is lying on his/her left side, or is lying on his/her face.

The posture measuring unit 30 may be implemented by manufacturing an acceleration sensor (a gravity sensor) in a form in which the acceleration sensor (the gravity sensor) may be attached to a user's (patient') body.

The determination unit 40 is a device determining whether or not a stroke occurs by receiving outputs of the left-side motion measurement unit 10, the right-side motion measurement unit 20, and the posture measurement unit 30 in a wired manner or a wireless manner.

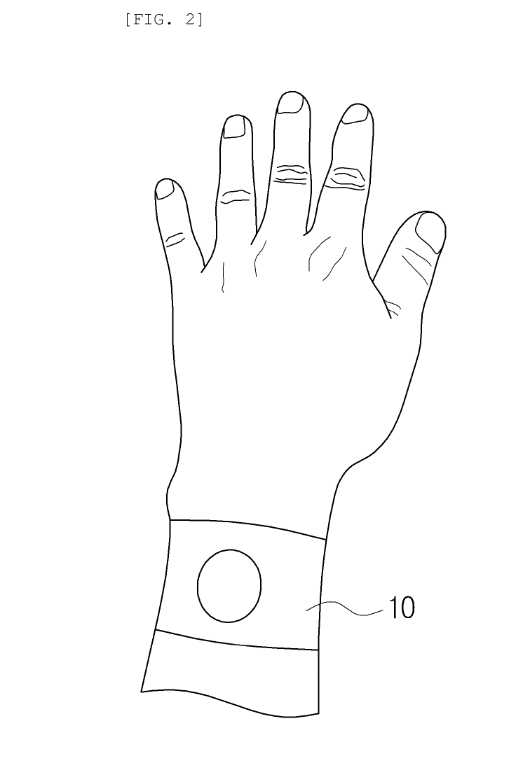

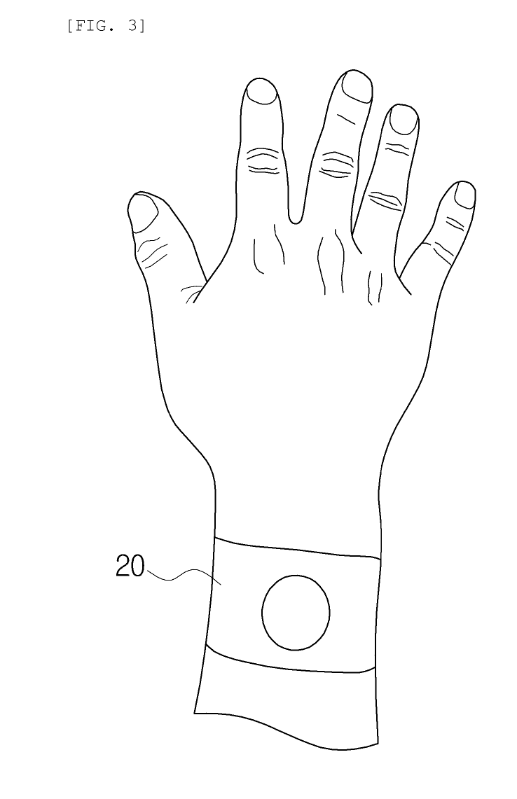

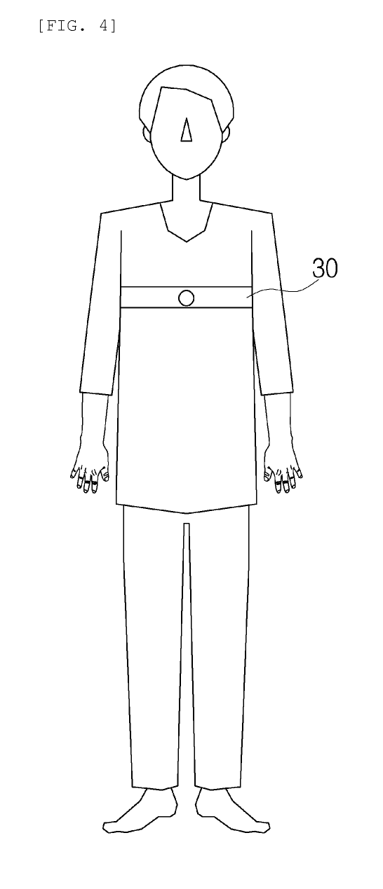

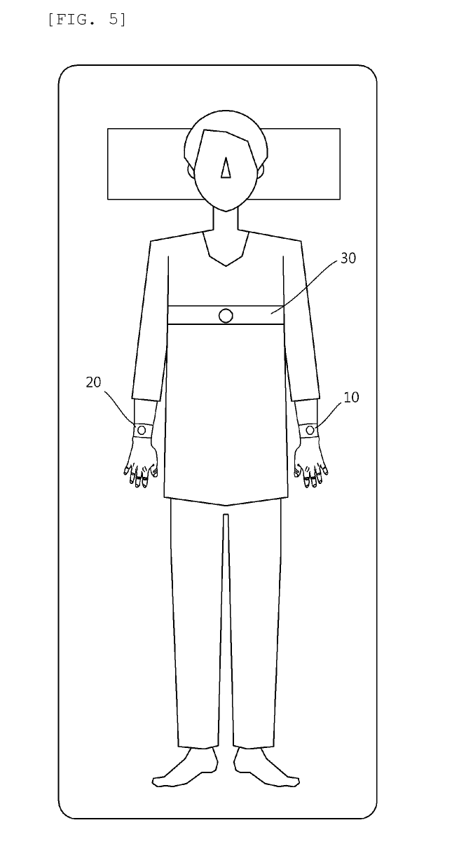

FIG. 2 is a view illustrating an example of a form in which a user wears a left-side motion measurement unit, FIG. 3 is a view illustrating an example of a form in which the user wears a right-side motion measurement unit, FIG. 4 is a view illustrating an example of a form in which the user wears a posture measurement unit, and FIG. 5 is a view illustrating a form in which the user wearing the left-side motion measurement unit, the right-side motion measurement unit, and the posture measurement unit is lying.

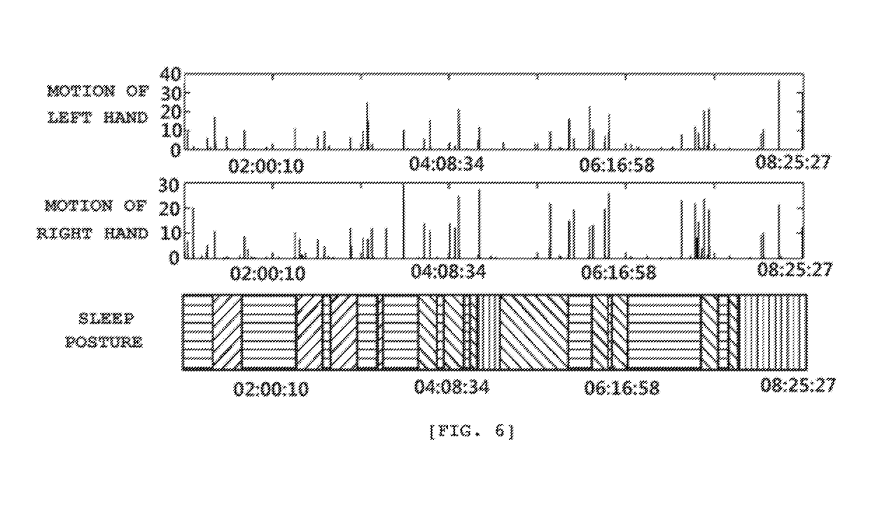

FIG. 6 is an example of measurement result graphs of a normal person. FIG. 6 illustrates an amount of motion of a right hand, an amount of motion of a left hand, and a sleep posture (a lying posture) depending on a change in a time.

An expression of the lying posture is changed depending on a time zone, and the change in the expression indicates that the lying posture is changed. Horizontally drawn lines indicate that the user is lying upward, vertically drawn lines indicate that the user is lying on his/her left side, oblique lines drawn from an upper portion of the right side to a lower portion of the left side indicates that the user is lying on his/her right side, and oblique lines drawn from an upper portion of the left side to a lower portion of the right side indicates that the user is lying on his/her face.

When lines drawn in different directions are expressed by colors, the horizontally drawn lines may be expressed by a red color, the vertically drawn lines may be expressed by a blue color, the oblique lines drawn from the upper portion of the right side to the lower portion of the left side may be expressed by a green color, and the oblique lines drawn from the upper portion of the left side to the lower portion of the right side may be expressed by a pink color.

Generally, in the early stage after a stroke occurs, hemiplegic symptoms in which one-side arm and one-side leg of the patient paralyzed appear. Therefore, motions of both arms and both legs of the patient are compared with each other, and when a motion of one-side arm and one-side leg of the patient is significantly less than that of the other-side arm and the other-side leg of the patient, a likelihood of the stroke is high. As a method for determining a stroke by comparing the motions of both arms and both legs of the patient with each other, there are several methods, and an example of the simplest method of them is a method of comparing amounts of motion during a unit time with each other and determining that the stroke occurs when a ratio between the amounts is larger than a predetermined reference value.

As a result of making the graphs of FIG. 6 with respect to many normal persons, in the case in which the user (the patient) is lying on his/her back (in the case in which the user (the patient) is lying with his/her breast facing upward), an amount of motion of a left hand and an amount of motion of a right hand is similar to each other, but during a period in which the user (the patient) is lying at another posture or changes his/her posture, even in the case of the normal person, a large difference is generated between an amount of motion of a left hand and an amount of motion of a right hand.

Therefore, if it is determined that the stroke occurs when a difference between the amount of motion of the left hand and the amount of motion of the right hand during a screening time (for example, a time from one hour prior to a determination point in time to the determination point in time) is a predetermined reference value or more, in the case in which the user (the patient) is continuously lying on his/her back, a determination result is relatively accurate, but when the user (the patient) is lying at another posture, even in the case of a normal state, it is recognized that a difference between left and right motions is large, such that it is likely that it will be determined that the stroke occurs.

Therefore, another determination reference needs to be applied during a period in which the user (the patient) is not continuously lying on his/her back (a period in which the user (the patient) is lying at another posture or changes his/her posture).

For example, the period in which the user (the patient) is not continuously lying on his/her back is excluded from the screening time, and the time from one hour prior to the determination point in time to the determination point in time in a time remaining after the exclusion is set to the screening time, and when the difference between the amount of motion of the left hand and the amount of motion of the right hand during the screening time is the predetermined reference value or more, it may be determined that the stroke occurs.

In addition, since a case in which the normal person continuously sleeps while maintaining a posture at which he/she lies on his/her left side, a posture at which he/she lies on his/her right side, or a posture at which he/she lies on his/her face hardly exists, when the user continuously sleeps while maintaining a posture at which he/she lies on his/her left side, a posture at which he/she lies on his/her right side, or a posture at which he/she lies on his/her face for a predetermined time (for example, two hours) or more, it may be determined that the stroke occurs.

In the graphs of FIG. 6, a process of deleting a result during the period in which the user (the patient) is not continuously lying on his/her back (the user (the patient) is lying at another posture or changes his/her posture may be considered as a process of passing through a sleeping position (SP) filter.

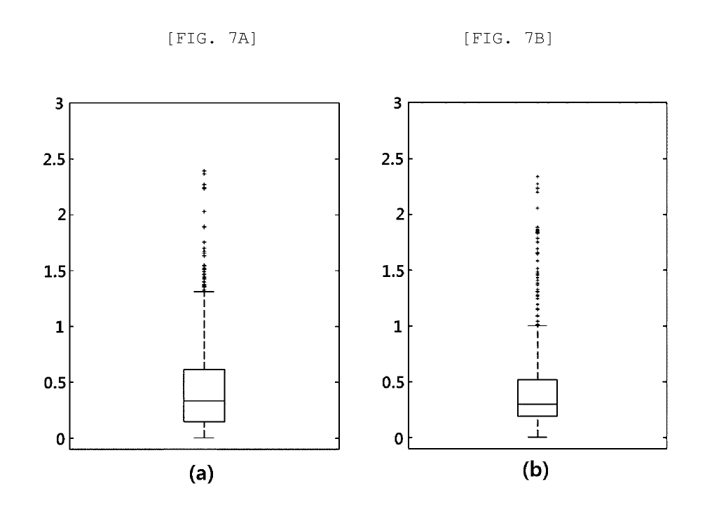

FIGS. 7A and 7B are views illustrating a form of comparing measurement result graphs depending on whether or not a sleeping position (SP) filter exists with each other.

FIG. 7A is a box plot of a ratio between motions when the SP filter does not exist, and FIG. 7B is a box plot of a ratio between motions when the SP filter exists.

A ratio between motions of both arms during the screening time may be calculated by the following Equation.

Ratio between motions=|log(Left value/Right value)|

That is, a logarithm value of a ratio between a motion of a left arm and a motion of a right arm may be calculated, and an absolute value of the logarithm value may be calculated, and the calculated absolute value may be considered as the ratio between the motions.

When the ratio between the motions is calculated several times, calculated values may be different from each other. Graphs illustrating the box plot of the ratio between the motions when the SP filter does not exist and the box plot of the ratio between the motions when the SP filter exists are illustrated in FIGS. 7A and 7B.

Referring to FIGS. 7A and 7B, it may be appreciated that when the SP filter exists, calculated values of the ratio between the motions are generally small.

Therefore, it may be appreciated that when the SP filter exists, even though the same reference value is applied, it is less likely to determine that the normal person is a stroke patient.

In order to a user's (patient's) posture, the posture measurement unit 30 attached to the user's body may be used. Alternatively, the user's posture may be estimated from outputs of the left-side motion measurement unit 10 and the right-side motion measurement unit 20 without installing a separate posture measurement unit 30.

There may be several methods for estimating the user's posture from the outputs of the left-side motion measurement unit 10 and the right-side motion measurement unit 20.

The simplest method of these methods is a method for measuring outputs of the left-side motion measurement unit 10 and the right-side motion measurement unit 20 for each posture at which the user lies, storing the measured outputs in a database, measuring actual outputs (outputs of the left-side motion measurement unit 10 and the right-side motion measurement unit 20) of the user, and comparing the actual outputs with the values stored in the database to find to output values of which posture the actual outputs are most similar.

A device estimating the user's posture from the outputs of the left-side motion measurement unit 10 and the right-side motion measurement unit 20 may be called a ‘posture estimation unit’. The posture estimation unit may be manufactured as a hardware separate from the determination unit or the determination unit may be configured to serve as the posture estimation unit.

A method for determining a stroke during sleep using the apparatus for determining a stroke during sleep according to the present invention including the left-side motion measurement unit, the right-side motion measurement unit, the posture measurement unit, and the determination unit may include the following steps.

First step: Step of receiving outputs of the left-side motion measurement unit, the right-side motion measurement unit, the posture measurement unit by the determination unit.

Second step: Step of applying the SP filter by the determination unit.

Third step: Step of determining whether or not the stroke occurs from a result obtained by applying the SP filter by the determination unit.

A method for determining a stroke during sleep using the apparatus for determining a stroke during sleep according to the present invention including the left-side motion measurement unit, the right-side motion measurement unit, the posture estimation unit, and the determination unit may include the following steps.

First step: Step of receiving outputs of the left-side motion measurement unit and the right-side motion measurement unit and then estimating the user's posture, by the posture estimation unit.

Second step: Step of receiving outputs of the left-side motion measurement unit, the right-side motion measurement unit, and the posture estimation unit by the determination unit.

Third step: Step of applying the SP filter by the determination unit.

Fourth step: Step of determining whether or not the stroke occurs from a result obtained by applying the SP filter by the determination unit.

A method for determining a stroke during sleep using the apparatus for determining a stroke during sleep according to the present invention including the left-side motion measurement unit, the right-side motion measurement unit, and the determination unit may include the following steps.

First step: Step of receiving outputs of the left-side motion measurement unit and the right-side motion measurement unit and then estimating the user's posture, by the determination unit.

Second step: Step of applying the SP filter on the basis of an estimation result by the determination unit.

Third step: Step of determining whether or not the stroke occurs from a result obtained by applying the SP filter by the determination unit.

The apparatus for determining a stroke during sleep according to the present invention may be connected to a personal communication apparatus (for example, a smartphone) in a wired manner or a wireless manner, and may transmit a signal for informing predefined emergency contact places (for example, 911, stroke hospitals, contact details of close relatives, and the like) of a stroke risk situation to the predefined emergency contact places through the personal communication apparatus in the case in which it is determined that a risk of the stroke is high.

Alternatively, in order to confirm whether or not the stroke occurs in a state in which the risk of the stroke is high, when the determination unit determines that the risk of the stroke is high, the apparatus for determining a stroke during sleep does not directly transmit the signal for informing the predefined emergency contact places (for example, 911, the stroke hospitals, the contact details of the close relatives, and the like) of the stroke risk situation to the predefined emergency contact places, but the personal communication apparatus may be set to first generate an alarm sound, and the apparatus for determining a stroke during sleep may determine that the stroke does not occur when the user of the apparatus for determining a stroke during sleep hears the alarm sound and then inputs an alarm release input to the personal communication apparatus by himself/herself, and may determine that the stroke occurs and transmit the signal for informing the predefined emergency contact places of the stroke risk situation to the predefined emergency contact places when the alarm release input is not input to the personal communication apparatus even after a predetermined time elapses.

In addition, the alarm sound is not implemented to be generated by the personal communication apparatus, but may be implemented to be generated by the apparatus for determining a stroke during sleep.

In addition, the determination unit of the apparatus for determining a stroke during sleep is not implemented by a separate hardware, but may be implemented using a hardware of the personal communication apparatus (for example, the smartphone). When the determination unit is configured as described above, some (the determination unit) of components of the apparatus for determining a stroke during sleep according to the present invention is positioned in the personal communication apparatus (for example, the smartphone), and the apparatus for determining a stroke during sleep according to the present invention may thus be considered as including the personal communication apparatus (for example, the smartphone).

1. An apparatus for determining a stroke during sleep, comprising:
a left-side motion measurement unit wearable on a left arm or a left leg;
a right-side motion measurement unit wearable on a right arm or a right leg;
a posture measurement unit wearable on a body; and
a determination unit receiving outputs of the left-side motion measurement unit, the right-side motion measurement unit, and the posture measurement unit and determining whether or not the stroke occurs.

2. The apparatus for determining a stroke during sleep of claim 1, wherein the determination unit applies a sleeping position (SP) filter (a processing process of deleting a measurement result when a user is not lying with his/her breast facing upward), and then compares a ratio between left and right motions to determine whether or not the stroke occurs.

3. The apparatus for determining a stroke during sleep of claim 2, wherein the determination unit determines that the stroke occurs in the case in which the user maintains a posture other than a posture at which he/she lies on his/her back, for a predetermined time or more.

4. An apparatus for determining a stroke during sleep, comprising:
a left-side motion measurement unit wearable on a left arm or a left leg;
a right-side motion measurement unit wearable on a right arm or a right leg;
a posture estimation unit receiving outputs of the left-side motion measurement unit and the right-side motion measurement unit and estimating a user's posture; and
a determination unit receiving outputs of the left-side motion measurement unit, the right-side motion measurement unit, and the posture estimation unit and determining whether or not the stroke occurs.

5. An apparatus for determining a stroke during sleep, comprising:
a left-side motion measurement unit wearable on a left arm or a left leg;
a right-side motion measurement unit wearable on a right arm or a right leg; and
a determination unit receiving outputs of the left-side motion measurement unit and the right-side motion measurement unit and determining whether or not the stroke occurs,
wherein the determination unit receives the outputs of the left-side motion measurement unit and the right-side motion measurement unit and estimates the user's posture, and then determines whether or not the stroke occurs using an estimation result.

6. A method for determining a stroke during sleep using an apparatus for determining a stroke during sleep including a left-side motion measurement unit, a right-side motion measurement unit, a posture measurement unit, and a determination unit, comprising:
a step of receiving outputs of the left-side motion measurement unit, the right-side motion measurement unit, the posture measurement unit by the determination unit;
a step of applying an SP filter by the determination unit; and
a step of determining whether or not the stroke occurs from a result obtained by applying the SP filter by the determination unit.

7. A method for determining a stroke during sleep using an apparatus for determining a stroke during sleep including a left-side motion measurement unit, a right-side motion measurement unit, a posture estimation unit, and a determination unit, comprising:
a step of receiving outputs of the left-side motion measurement unit and the right-side motion measurement unit and then estimating a user's posture, by the posture estimation unit;
a step of receiving outputs of the left-side motion measurement unit, the right-side motion measurement unit, and the posture estimation unit by the determination unit; and
a step of applying an SP filter by the determination unit; and
a step of determining whether or not the stroke occurs from a result obtained by applying the SP filter by the determination unit.

8. A method for determining a stroke during sleep using an apparatus for determining a stroke during sleep including a left-side motion measurement unit, a right-side motion measurement unit, and a determination unit, comprising:
a step of receiving outputs of the left-side motion measurement unit and the right-side motion measurement unit and then estimating a user's posture, by the determination unit;
a step of applying an SP filter on the basis of an estimation result by the determination unit; and
a step of determining whether or not the stroke occurs from a result obtained by applying the SP filter by the determination unit.
